(19) United States
(12) Patent Application Publication
(10) Pub. No.: US 2026/0117257 A1
(43) Pub. Date: Apr. 30, 2026

(54) MRNA Delivery System Targeting DC Cells and Preparation Method Therefor

(71) Applicants: Winlein (Tianjin) Biotechnology Co., Ltd., Tianjin (CN); NATIONAL UNIVERSITY CORPORATION KUMAMOTO UNIVERSITY, Kumamoto-shi (JP)

(72) Inventors: Kunpeng Li, Tianjin (CN); Taishi Higashi, Kumamoto-shi (JP)

(21) Appl. No.: 19/371,310

(22) Filed: Oct. 28, 2025

(30) Foreign Application Priority Data
Oct. 29, 2024 (CN) 202411516828.2

(51) Int. Cl. C12N 15/88

(52) U.S. Cl. CPC C12N 15/88

(57) ABSTRACT

A mRNA delivery system targeting DC cells (Dendritic Cells) and a preparation method therefor are provided, relating to the technical field of biomedicals. Two molecules, mannose and glycocyamine, are innovatively grafted onto a cyclodextrin structure of polyrotaxane via triethylenetetramine. The grafted mannose has a DC cell targeting effect, while a guanidine group from the glycocyamine has potential cell penetration. An experimental result shows that when the mannose and glycocyamine are simultaneously grafted onto the cyclodextrin structure of polyrotaxane, the targeting accuracy and delivery efficiency of the delivery system to the DC cells can be synergistically improved. The prepared mRNA delivery system that targets DC cells not only enriches the research scope of a nucleic acid delivery system, but also provides a strong technical support for biomedical fields such as tumor therapy.

### TECHNICAL FIELD

The present disclosure belongs to the technical field of biomedicals, and in particular to a mRNA delivery system targeting DC cells (Dendritic Cells) and a preparation method therefor.

### BACKGROUND

In organisms, due to their inherent susceptibility to degradation, nucleic acids make efficient delivery systems an indispensable key element in the development of pharmaceuticals. In 2020, BioNTech in Germany, along with Pfizer and Moderna in USA, by virtue of innovative mRNA vaccine technology and a meticulously constructed LNP delivery system composed of four components including liposomes, has successfully encapsulated mRNA and safely delivered the mRNA into the human body, opening a new chapter in the field of nucleic acid therapy.

In the field of tumor therapy, mRNA-based therapeutic tumor vaccines, with their ability to accurately translate tumor-specific antigens, exhibit higher accuracy and simplified quality control processes compared to conventional methods. However, to further enhance the therapeutic efficacy, the antigens need to be precisely delivered into specific cells such as dendritic cells (DC cells). Unfortunately, although the LNP and other delivery systems have dominated the market, targeted nucleic acid tumor vaccine delivery platforms specifically for DC cells remains a blank area, and there is an urgent need to develop solutions that are both safe and efficient.

Polyrotaxane, a molecular structure ingeniously formed by the combination of cyclic molecules and chain-like molecules, derives its unique dynamic reversibility from the free sliding and rotation of cyclodextrins along the polymer chain, which endows the material with unprecedented molecular mobility. In the field of biomedical engineering, due to this property, the polyrotaxane not only can optimize initial response performance of the material, but also can enhance interactions with target objects such as drugs, genes, and cells through multi-site synergistic effects, and further can affect functions of the target objects and even the construction of the cytoskeleton, demonstrating a broad application prospect. The potential of the polyrotaxane to promote stable introduction of functional nucleic acids and proteins into Hela cells has been disclosed in Patent CN117203244A, and efficient intracellular delivery is achieved by specific modification.

Basic amino acids such as arginine have long been regarded as effective means for enhancing cell penetration due to their unique chemical structures and positive charge properties. Arginine can promote molecules to cross the cell membrane barrier and achieve intracellular delivery through interaction with negative charges on the cell membrane surface. However, as research advances, scientists continue to explore new cell-penetrating peptides, aiming to optimize biocompatibility and reduce potential toxicity while improving penetration efficiency. Glycocyamine, a compound conventionally and widely used in pharmaceutical organic synthesis and as a food and feed additive, is rarely applied in the biomedical field. Based on the foregoing background, the present disclosure provides a mRNA delivery system targeting DC cells and a preparation method for the mRNA delivery system. By grafting mannose and glycocyamine onto cyclodextrin of polyrotaxane, the targeting accuracy and delivery efficiency of the delivery system to the DC cells can be synergistically improved.

### SUMMARY

A first objective of the present disclosure provides a mRNA delivery system targeting DC cells, where a structural formula of the delivery system is as shown in formula I.

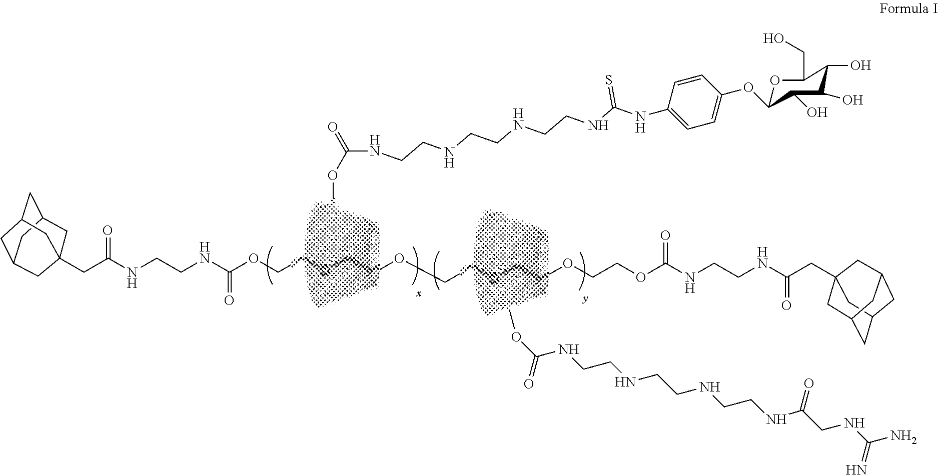

where x is an integer selected from 5 to 10, and y is an integer selected from 5 to 10.

A second objective of the present disclosure is to provide a preparation method for the mRNA delivery system targeting DC cells, including the following steps:

(1) preparing ethylenediamine-modified polyethylene glycol: mixing polyethylene glycol with N,N′-carbonyldiimidazole, stirring at 50-55° C. for 16-20 h under nitrogen protection, and then adding ethylenediamine to react for 2-3 h, after the reaction is finished, adding ethanol to a reaction mixture, standing, collecting precipitate, washing and drying the precipitate to obtain NH₂—PEG-NH₂;

(2) preparing pseudopolyrotaxane: adding NH₂—PEG-NH₂ to a cyclodextrin aqueous solution to react, after the reaction is finished, collecting precipitate, and freeze-drying the precipitate to obtain PPR;

(3) preparing polyrotaxane: mixing PPR with a capping agent, and then adding benzotriazol-1-yloxytris(dimethylamino) phosphonium hexafluorophosphate, 1-hydroxybenzotriazole and 1,1′-[(2-hydroxyethyl)imino]dipropan-2-ol, stirring and reacting at 4-5° C. for 40-50 h under a nitrogen atmosphere, and after the reaction is finished, collecting precipitate, washing and drying the precipitate to obtain PRX;

(4) preparing urethane-modified polyrotaxane: mixing PRX with N,N′-carbonyldiimidazole, and adding triethylenetetramine for reaction, and after the reaction is finished, carrying out dialysis, and freeze-drying to obtain PRX-TETA;

(5) preparing glycocyamine-modified polyrotaxane: adding PRX-TETA to DMF (N,N-Dimethylformamide), adding Pbf-protected glycocyamine, 1-ethyl-3-(3-dimethylaminopropyl)carbodiimide hydrochloride, 2,2-bis(hydroxymethyl)propionic acid, 1-hydroxybenzotriazole and N,N-diisopropylethylamine, stirring and reacting for 12-15 h, and after the reaction is finished, centrifuging, collecting precipitate, and washing and drying the precipitate to obtain Pbf-Ga-PRX;

(6) preparing mannose-modified polyrotaxane: mixing Pbf-Ga-PRX with 4-isothiocyanatophenyl-α-D-mannoside, stirring and reacting for 24-32 h, and after the reaction is finished, carrying out dialysis, and freeze-drying to obtain Pbf-Ga-PRX-Man; and

(7) preparing a mRNA delivery system targeting DC cells: adding Pbf-Ga-PRX-Man and trifluoroacetic acid into DMF, stirring and reacting for 0.5-1 h, adding diethyl ether to a reaction system, filtering, collecting precipitate, and drying the precipitate to obtain the mRNA delivery system targeting DC cells.

Further, in step (1), a mole ratio of polyethylene glycol to N,N′-carbonyldiimidazole to ethylenediamine is 1:(4.5-5):(32-33).

Further, in step (2), a dosage ratio of NH₂-PEG-NH₂ to the cyclodextrin aqueous solution is 1 g:(30-40) mL; and a mass concentration of the cyclodextrin aqueous solution is 12%-13%.

Further, in step (3), a mass ratio of PPR to the capping agent to the benzotriazol-1-yloxytris(dimethylamino) phosphonium hexafluorophosphate to 1-hydroxybenzotriazole is (5.5-5.6):1:(2.0-2.5):(0.7-1.0), and a dosage ratio of the capping agent to the 1,1′-[(2-hydroxyethyl)imino]dipropan-2-ol is 1 g:(0.9-1.0) mL.

Further, in step (4), a mole ratio of PRX to N,N′-carbonyldiimidazole to triethylenetetramine is 1:(20-50):(50-80).

Further, in step (5), a mole ratio of PRX-TETA to Pbf-protected glycocyamine to 1-ethyl-3-(3-dimethylaminopropyl)carbodiimide hydrochloride to 2,2-bis(hydroxymethyl)propionic acid to 1-hydroxybenzotriazole to N,N-diisopropylethylamine is 1:(5-10):(1-1.2):(1-1.2):(0.05-0.1):(5-5.5).

Further, in step (6), a mole ratio of Pbf-Ga-PRX to 4-isothiocyanatophenyl-α-D-mannoside is 1:(5-10).

Further, in step (7), a dosage ratio of Pbf-Ga-PRX-Man to trifluoroacetic acid is 1 mg:(0.9-1.5) mL.

Further, the capping agent in step (3) is adamantaneacetic acid.

Compared with the prior art, the present disclosure has beneficial effects as follows.

(1) Two molecules, mannose and glycocyamine, are innovatively grafted onto a cyclodextrin structure of polyrotaxane via triethylenetetramine. The grafted mannose has a DC cell targeting effect, while a guanidine group from the glycocyamine has potential cell penetration. An experimental result shows that when the mannose and glycocyamine are simultaneously grafted onto the cyclodextrin structure of polyrotaxane, the targeting accuracy and delivery efficiency of the delivery system to the DC cells can be synergistically improved.

(2) The prepared mRNA delivery system that targets DC cells not only enriches the research scope of a nucleic acid delivery system, but also provides a strong technical support for biomedical fields such as tumor therapy.

### BRIEF DESCRIPTION OF THE DRAWINGS

FIG. 1 is a flowchart of preparation of a mRNA delivery system targeting DC cells according to the present disclosure;

FIG. 2 is a ¹H-NMR (Proton Nuclear Magnetic Resonance) diagram of NH₂-PEG-NH₂ according to the present disclosure;

FIG. 3 is a ¹H-NMR diagram of PPR according to the present disclosure;

FIG. 4 is a ¹H-NMR diagram of PRX-TETA according to the present disclosure;

FIG. 5 is a ¹H-NMR diagram of Pbf-Ga-TETA-PRX according to the present disclosure;

FIG. 6 is a ¹H-NMR diagram of a mRNA delivery system targeting DC cells according to the present disclosure;

FIG. 7 is a confocal laser scanning microscope image of cells in each group in Experimental Example 1 of the present disclosure.

### DETAILED DESCRIPTION OF THE EMBODIMENTS

The technical solutions of the present disclosure are further explained and described below with reference to specific embodiments, comparative examples and experiment examples.

In the following embodiments, comparative examples, and experimental examples, unless otherwise specified, the raw materials and preparation methods used are conventional materials and techniques in the field.

A preparation method for Pbf-Ga in step (5) in the following embodiment is as follows.

(1) 5 mL of thionyl chloride was added to 50 mL of absolute ethyl alcohol at −5° C., and then 0.05 mol of glycocyamine was added to obtain a mixture, and then a mixture was heated to a room temperature and allowed to react for 48 h. After the reaction was finished, concentration was carried out under a reduced pressure to obtain ethyl guanidinoacetate.

(2) The ethyl guanidinoacetate was added to 100 mL of acetone, and then 2,2,4,6,7-pentamethyldihydrobenzofuran-5-sulfonyl chloride (Pbf-Cl, 0.055 mol) and potassium carbonate (0.15 mol) were added to obtain a mixture, and then the mixture was stirred at 40-45° C. The reaction was monitored by TLC. After the complete reaction of the ethyl guanidinoacetate, suction filtration was carried out to obtain a filtrate, and the filtrate was subjected to vacuum distillation to obtain Pbf-protected ethyl guanidinoacetate.

(3) The Pbf-protected ethyl guanidinoacetate was added to 50 mL of ethanol with a concentration of 75%, and NaOH aqueous solution was added dropwise to regulate pH to 11, and a hydrolysis reaction was carried out at a room temperature. After the hydrolysis reaction was finished, pH of a reaction mixture was regulated to 7 with HCl, and then the reaction mixture was cooled to 0° C. for crystallization and centrifuged for solid collection, where the collected solid was washed with ethyl acetate and dried to obtain Pbf-protected glycocyamine, which was named Pbf-Ga.

### Embodiment 1

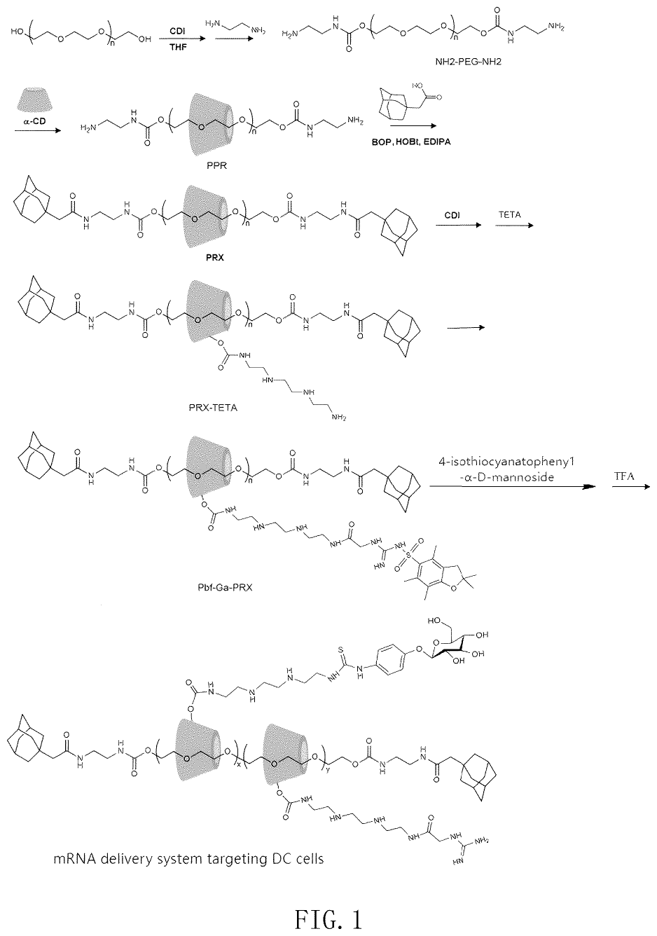

A preparation flow of a mRNA delivery system targeting DC cells is as shown in FIG. 1, specifically including the following steps.

### (1) Preparation of Ethylenediamine-Modified Polyethylene Glycol

1 mmol of PEG (Polyethylene Glycol) (20 kDa) was added to 100 mL of THF (Tetrahydrofuran), and then 4.5 mmol of N,N′-carbonyldiimidazole (CDI) was added to obtain a mixture, and then the mixture was stirred at 50° C. for 16 h under a nitrogen atmosphere. Afterwards, 32 mmol of ethylenediamine was added into the reaction mixture to react at 50° C. for 2 h. After the reaction was finished, 100 mL of ethanol was added to the reaction mixture, which was then allowed to stand at −20° C. for 2 h. The mixture was centrifuged for precipitate collection. The precipitate was washed with ethanol and then dried to obtain ethylenediamine-modified polyethylene glycol, which was named NH₂-PEG-NH₂.

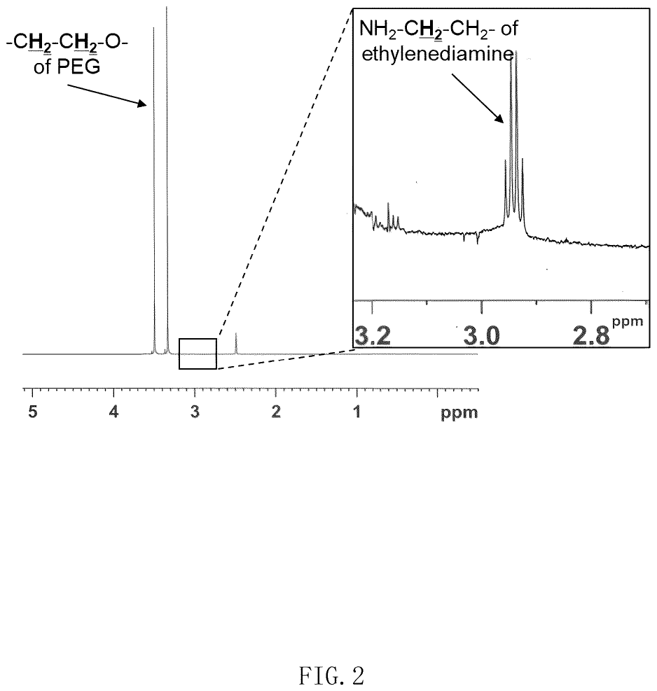

A ¹H-NMR diagram of NH₂-PEG-NH₂ is shown in FIG. 2.

### (2) Preparation of Pseudopolyrotaxane

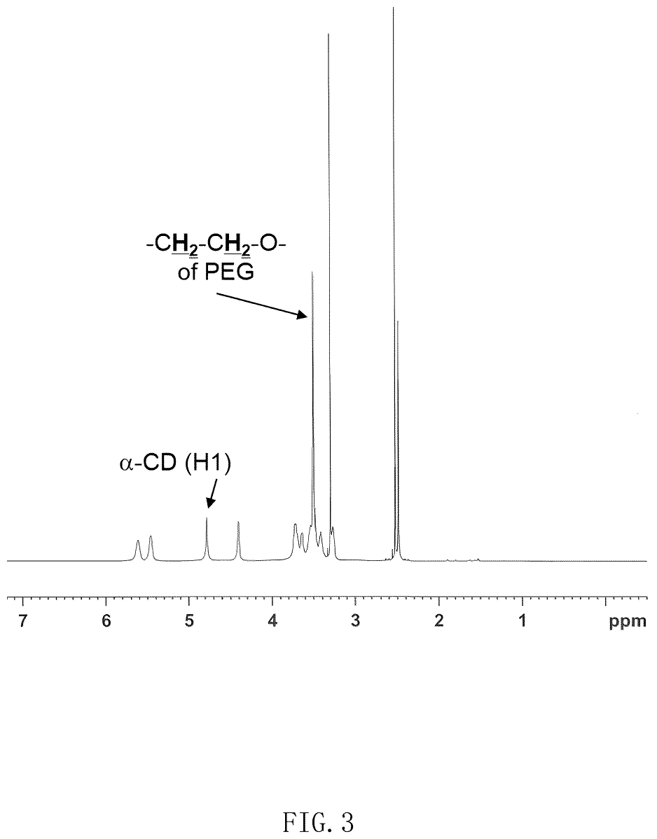

6 g of NH₂—PEG-NH₂ was added to 200 mL of α-cyclodextrin aqueous solution with a concentration of 12% (w/v) to obtain a mixture, the mixture was stirred at 4° C. for one night and centrifuged, and precipitate was collected and freeze-dried to obtain pseudopolyrotaxane, which was named PPR. A ¹H-NMR diagram of PPR is as shown in FIG. 3.

### (3) Preparation of Polyrotaxane

1 g of adamantaneacetic acid, 2 g of BOP (benzotriazol-1-yloxytris(dimethylamino) phosphonium hexafluorophosphate), 0.7 g of 1-hydroxybenzotriazole (HOBt) and 0.9 mL of 1,1′-[(2-hydroxyethyl)imino]dipropan-2-ol (EDIPA) were added to 100 mL of DMF, and after dissolution, 5.5 g of PPR was added to obtain a mixture, and the mixture was stirred and allowed to react at 4° C. for 40 h under a nitrogen atmosphere. After the reaction was finished, the mixture was centrifuged for precipitate collection. The precipitate was washed twice with an ethanol/DMF mixed solvent (v:v=1:1), the washed precipitate was added to DMSO for dissolution and was re-precipitated by dropwise adding cold water therein. The operation was repeated for three times to obtain precipitate, and then the precipitate was freeze-dried to obtain polyrotaxane, which was named PRX.

### (4) Preparation of Urethane-Modified Polyrotaxane

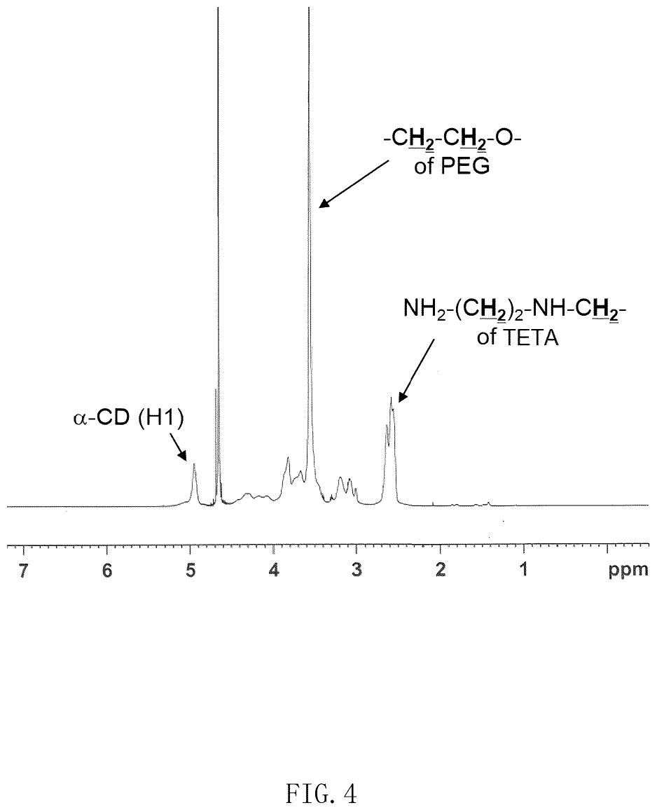

50 mg of PRX was added to 15 mL of DMSO, and CDI was added to obtain a mixture, after the mixture was stirred at a room temperature for one night under a nitrogen atmosphere, triethylene tetramine (TETA) was added to a reaction mixture, and the reaction mixture was continuously stirred at the room temperature for one night, where a mole ratio to PRX to CDI to TETA was 1:30:60. After the reaction was finished, dialysis (Spectra/Por @Membrane, MWCO: 10 kDa) was carried out in pure water to remove unreacted CDI and TETA, and then freeze-drying was carried out to obtain urethane-modified polyrotaxane, which was named PRX-TETA. A ¹H-NMR diagram of PRX-TETA is as shown in FIG. 4.

### (5) Preparation of Glycocyamine-Modified Polyrotaxane:

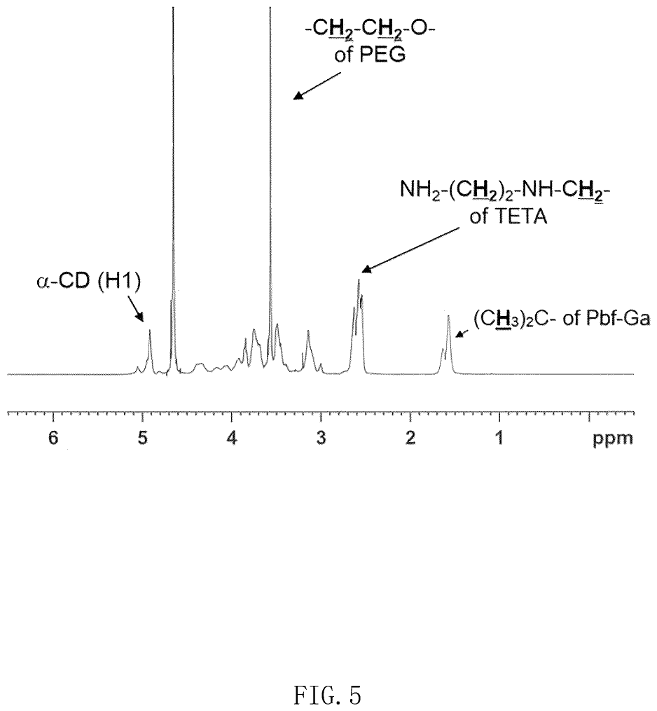

80 mg of PRX-TETA obtained in step (4) was added to 50 mL of DMF, Pbf-protected glycocyamine (Pbf-Ga), 1-ethyl-3-(3-dimethylaminopropyl)carbodiimide hydrochloride (EDC), 2,2-bis(hydroxymethyl)propionic acid (DMPA), HOBt and N,N-diisopropylethylamine (DIPEA) were added to obtain a mixture, then the mixture was stirred and allowed to react at a room temperature for 12 h, where a mole ratio of PRX-TETA to Pbf-Ga to EDC to DMPA to HOBt to DIPEA was 1:5:1:1:0.05:5. After the reaction was finished, the mixture was centrifuged for precipitate collection. The precipitate was washed twice with an ethanol/DMF mixed solvent (v:v=1:1), the washed precipitate was added to DMSO for dissolution, and was re-precipitated by dropwise adding cold water therein. The operation was repeated for three times to obtain precipitate, and then the precipitate was freeze-dried to obtain glycocyamine-modified polyrotaxane, which was named Pbf-Ga-PRX. A ¹H-NMR diagram of Pbf-Ga-PRX is as shown in FIG. 5.

### (6) Preparation of Mannose-Modified Polyrotaxane:

Pbf-Ga-PRX was added to a mixed solution of 5.0 mL of NaHCO₃ buffer solution and 16 mL of DMSO for dissolution to obtain Pbf-Ga-PRX solution for further use.

4-isothiocyanatophenyl-α-D-mannoside was dissolved with 10 mL of DMSO to obtain a mixture, Pbf-Ga-PRX solution was dropwise added into the mixture according to a mole ratio of Pbf-Ga-PRX to 4-isothiocyanatophenyl-α-D-mannoside of 1:5, and then the mixture was stirred at a room temperature for 24 h. After the reaction was finished, dialysis (Spectra/Por® Membrane, MWCO: 10 kDa, solvent:water) was carried out to remove unreacted 4-isothiocyanatophenyl-α-D-mannoside, and then freeze-drying was carried out to obtain mannose-modified polyrotaxane, which was named Pbf-Ga-PRX-Man.

(7) Preparation of mRNA Delivery System Targeting DC Cells

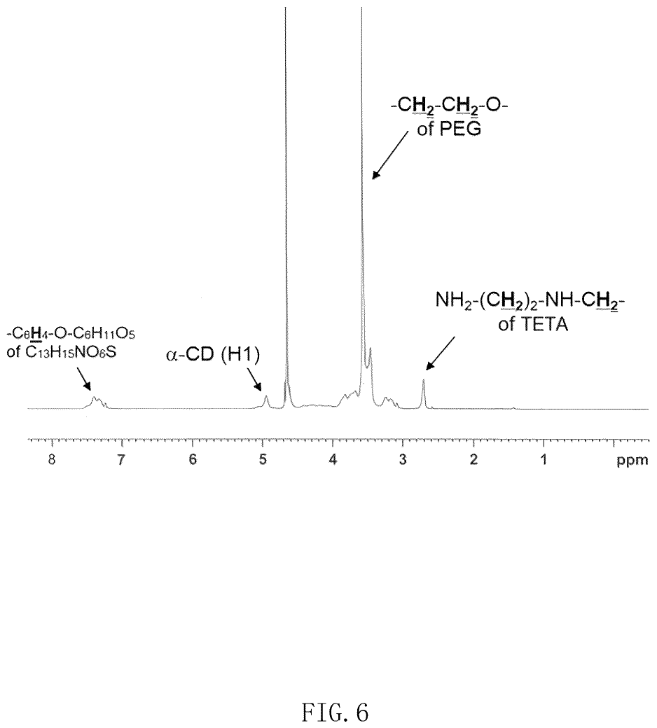

30 mg of Pbf-Ga-PRX-Man was added to 30 mL of anhydrous DMF, and 45 mL of trifluoroacetic acid was added to obtain a mixture, then the mixture was stirred at 200 r/min and allowed to react for 30 min, 4° C. anhydrous diethyl ether was added until a large amount of precipitate was formed. The precipitate was obtained by suction filtration, dissolved in 15 mL of anhydrous DMF, and subjected to an ether precipitation method for three times. Then, the precipitate was dried to obtain a mRNA delivery system targeting DC cells, which was named C1. A ¹H-NMR diagram of C1 is as shown in FIG. 6.

### Embodiment 2

A mRNA delivery system targeting DC cells specifically includes the following steps.

### (1) Preparation of Ethylenediamine-Modified Polyethylene Glycol

1 mmol of PEG (20 kDa) was added to 100 mL of THF, and then 5 mmol of N,N′-carbonyldiimidazole (CDI) was added to obtain a mixture, and then the mixture was stirred at 50° C. for 20 h under a nitrogen atmosphere. Afterwards, 33 mmol of ethylenediamine was added into the reaction mixture to react at 50° C. for 3 h. After the reaction was finished, 100 mL of ethanol was added to the reaction mixture, which was then allowed to stand at −20° C. for 2 h. The mixture was centrifuged for precipitate collection. The precipitate was washed with ethanol and then dried to obtain ethylenediamine-modified polyethylene glycol, which was named NH₂-PEG-NH₂.

### (2) Preparation of Pseudopolyrotaxane

6 g of NH₂—PEG-NH₂ was added to 250 mL of α-cyclodextrin aqueous solution with a concentration of 13% (w/v) to obtain a mixture, the mixture was stirred at 4° C. for one night and centrifuged, and precipitate was collected and freeze-dried to obtain pseudopolyrotaxane, which was named PPR.

### (3) Preparation of Polyrotaxane

1 g of adamantaneacetic acid, 2.5 g of BOP, 1.0 g of HOBt and 1.0 mL of 1,1′-[(2-hydroxyethyl)imino]dipropan-2-ol (EDIPA) were added to 100 mL of DMF, and after dissolution, 5.6 g of PPR was added to obtain a mixture, and the mixture was stirred and allowed to react at 5° C. for 50 h under a nitrogen atmosphere. After the reaction was finished, the mixture was centrifuged for precipitate collection. The precipitate was washed twice with an ethanol/DMF mixed solvent (v:v=1:1), the washed precipitate was added to DMSO for dissolution and was re-precipitated by dropwise adding cold water therein. The operation was repeated for three times to obtain precipitate, and then the precipitate was freeze-dried to obtain polyrotaxane, which was named PRX.

### (4) Preparation of Urethane-Modified Polyrotaxane

50 mg of PRX was added to 15 mL of DMSO, and CDI was added to obtain a mixture, after the mixture was stirred at a room temperature for one night under a nitrogen atmosphere, TETA was added to a reaction mixture, and the reaction mixture was continuously stirred at the room temperature for one night, where a mole ratio to PRX to CDI to TETA was 1:20:80. After the reaction was finished, dialysis (Spectra/Por® Membrane, MWCO: 10 kDa) was carried out in pure water to remove unreacted CDI and TETA, and then the reaction mixture was freeze-dried to obtain urethane-modified polyrotaxane, which was named PRX-TETA.

### (5) Preparation of Glycocyamine-Modified Polyrotaxane

80 mg of PRX-TETA was added to 20 mL of DMF, Pbf-Ga, EDC, DMPA, HOBt and DIPEA were added to obtain a mixture, then the mixture was stirred and allowed to react at a room temperature for 15 h, where a mole ratio of PRX-TETA to Pbf-Ga to EDC to DMPA to HOBt to DIPEA was 1:8:1.2:1.2:0.1:5.5. After the reaction was finished, the mixture was centrifuged for precipitate collection. The precipitate was washed twice with an ethanol/DMF mixed solvent (v:v=1:1), the washed precipitate was added to DMSO for dissolution, and was re-precipitated by dropwise adding cold water therein. The operation was repeated for three times to obtain precipitate, and then the precipitate was freeze-dried to obtain glycocyamine-modified polyrotaxane, which was named Pbf-Ga-PRX.

### (6) Preparation of Mannose-Modified Polyrotaxane

80 mg of Pbf-Ga-PRX was added to a mixed solution of 5.0 mL of NaHCO₃ buffer solution and 16 mL of DMSO for dissolution to obtain Pbf-Ga-PRX solution for further use. 4-isothiocyanatophenyl-α-D-mannoside was dissolved with 10 mL of DMSO to obtain a mixture, Pbf-Ga-PRX solution was dropwise added into the mixture according to a mole ratio of Pbf-Ga-PRX to 4-isothiocyanatophenyl-α-D-mannoside of 1:8, and then the mixture was stirred at a room temperature for 24 h. After the reaction was finished, dialysis (Spectra/Por® Membrane, MWCO: 10 kDa, solvent:water) was carried out to remove unreacted 4-isothiocyanatophenyl-α-D-mannoside, and then freeze-drying was carried out to obtain mannose-modified polyrotaxane, which was named Pbf-Ga-PRX-Man.

(7) Preparation of mRNA Delivery System Targeting DC Cells

50 mg of Pbf-Ga-PRX-Man was added to 50 mL of anhydrous DMF, and 45 mL of trifluoroacetic acid was added to obtain a mixture, then the mixture was stirred at 200 r/min and allowed to react for 1 h, 4° C. anhydrous diethyl ether was added until a large amount of precipitate was formed. The precipitate was obtained by suction filtration, dissolved in 15 mL of anhydrous DMF, and subjected to an ether precipitation method for three times. Then, the precipitate was dried to obtain a mRNA delivery system targeting DC cells.

### Embodiment 3

A mRNA delivery system targeting DC cells specifically includes the following steps.

### (1) Preparation of Ethylenediamine-Modified Polyethylene Glycol

1 mmol of PEG (20 kDa) was added to 100 mL of THF, and then 5 mmol of N,N′-carbonyldiimidazole (CDI) was added to obtain a mixture, and then the mixture was stirred at 50° C. for 18 h under a nitrogen atmosphere. Afterwards, 33 mmol of ethylenediamine was added into the reaction mixture to react at 50° C. for 3 h. After the reaction was finished, 100 mL of ethanol was added to the reaction mixture, which was then allowed to stand at −20° C. for 2 h. The mixture was centrifuged for precipitate collection. The precipitate was washed with ethanol and then dried to obtain ethylenediamine-modified polyethylene glycol, which was named NH₂-PEG-NH₂.

### (2) Preparation of Pseudopolyrotaxane

5 g of NH₂-PEG-NH₂ was added to 250 mL of α-cyclodextrin aqueous solution with a concentration of 13% (w/v) to obtain a mixture, the mixture was stirred at 4° C. for one night and centrifuged, and precipitate was collected and freeze-dried to obtain pseudopolyrotaxane, which was named PPR.

### (3) Preparation of Polyrotaxane

1 g of adamantaneacetic acid, 2.5 g of BOP, 0.8 g of HOBt and 1.0 mL of 1,1′-[(2-hydroxyethyl)imino]dipropan-2-ol (EDIPA) were added to 100 mL of DMF, and after dissolution, 5.6 g of PPR was added to obtain a mixture, and the mixture was stirred and allowed to react at 5° C. for 50 h under a nitrogen atmosphere. After the reaction was finished, the mixture was centrifuged for precipitate collection. The precipitate was washed twice with an ethanol/DMF mixed solvent (v:v=1:1), the washed precipitate was added to DMSO for dissolution and was re-precipitated by dropwise adding cold water therein. The operation was repeated for three times to obtain precipitate, and then the precipitate was freeze-dried to obtain polyrotaxane, which was named PRX.

### (4) Preparation of Urethane-Modified Polyrotaxane

50 mg of PRX was added to 15 mL of DMSO, and CDI was added to obtain a mixture, after the mixture was stirred at a room temperature for one night under a nitrogen atmosphere, TETA was added to a reaction mixture, and the reaction mixture was continuously stirred at the room temperature for one night, where a mole ratio to PRX to CDI to TETA was 1:20:80. After the reaction was finished, dialysis (Spectra/Por® Membrane, MWCO: 10 kDa) was carried out in pure water to remove unreacted CDI and TETA, and then the reaction mixture was freeze-dried to obtain urethane-modified polyrotaxane, which was named PRX-TETA.

### (5) Preparation of Glycocyamine-Modified Polyrotaxane

80 mg of PRX-TETA was added to 20 mL of DMF, Pbf-Ga, EDC, DMPA, HOBt and DIPEA were added to obtain a mixture, then the mixture was stirred and allowed to react at a room temperature for 15 h, where a mole ratio of PRX-TETA to Pbf-Ga to EDC to DMPA to HOBt to DIPEA was 1:10:1.2:1.2:0.1:5.5. After the reaction was finished, the mixture was centrifuged for precipitate collection. The precipitate was washed twice with an ethanol/DMF mixed solvent (v:v=1:1), the washed precipitate was added to DMSO for dissolution, and was re-precipitated by dropwise adding cold water therein. The operation was repeated for three times to obtain precipitate, and then the precipitate was freeze-dried to obtain glycocyamine-modified polyrotaxane, which was named Pbf-Ga-PRX.

### (6) Preparation of Mannose-Modified Polyrotaxane

80 mg of Pbf-Ga-PRX was added to a mixed solution of 5.0 mL of NaHCO₃ buffer solution and 16 mL of DMSO for dissolution to obtain Pbf-Ga-PRX solution for further use. 4-isothiocyanatophenyl-α-D-mannoside was dissolved with 10 mL of DMSO to obtain a mixture, Pbf-Ga-PRX solution was dropwise added into the mixture according to a mole ratio of Pbf-Ga-PRX to 4-isothiocyanatophenyl-α-D-mannoside of 1:10, and then the mixture was stirred at a room temperature for 24 h. After the reaction was finished, dialysis (Spectra/Por® Membrane, MWCO: 10 kDa, solvent:water) was carried out to remove unreacted 4-isothiocyanatophenyl-α-D-mannoside, and then freeze-drying was carried out to obtain mannose-modified polyrotaxane, which was named Pbf-Ga-PRX-Man.

(7) Preparation of mRNA Delivery System Targeting DC Cells

50 mg of Pbf-Ga-PRX-Man was added to 50 mL of anhydrous DMF, and 50 mL of trifluoroacetic acid was added to obtain a mixture, then the mixture was stirred at 200 r/min and allowed to react for 1 h, 4° C. anhydrous diethyl ether was added until a large amount of precipitate was formed. The precipitate was obtained by suction filtration, dissolved in 15 mL of anhydrous DMF, and subjected to an ether precipitation method for three times. Then, the precipitate was dried to obtain a mRNA delivery system targeting DC cells.

### Comparative Example 1

Comparative Example 1 provides a mRNA delivery system, a preparation method for the mRNA delivery system is basically the same as that in Embodiment 1, and the difference from Embodiment 1 is that step (5) and step (7) are omitted in Comparative Example 1, and step (6) is as follows:

PRX-TETA was added to a mixed solution of 5.0 mL of NaHCO₃ buffer solution and 16 mL of DMSO for dissolution to obtain PRX-TETA solution for further use.

4-isothiocyanatophenyl-α-D-mannoside and glycocyamine were dissolved with 10 mL of DMSO to obtain a mixture, PRX-TETA solution was dropwise added to the mixture according to a mole ratio of PRX-TETA to glycocyamine to 4-isothiocyanatophenyl-α-D-mannoside of 1:5:5, and then the mixture was stirred at a room temperature for 24 h. After the reaction was finished, the reaction mixture was freeze-dried to obtain a mRNA delivery system, which was named D1.

### Comparative Example 2

Comparative Example 2 provides a mRNA delivery system, a preparation method for the mRNA delivery system is basically the same as that in Embodiment 1, and the difference from Embodiment 1 is that step (5) and step (7) are omitted in Comparative Example 2, and Pbf-Ga-PRX in step (6) is replaced with PRX-TET. A finally obtained mRNA delivery system was named D2.

### Comparative Example 3

Comparative Example 3 provides a mRNA delivery system, a preparation method for the mRNA delivery system is basically the same as that in Embodiment 1, the difference from Embodiment 1 is that Pbf-Ga in step (5) in Embodiment 1 is replaced with N-Boc-N′-(2,2,4,6,7-pentamethyldihydrobenzofuran-5-sulfonyl)-L-arginine. A finally obtained mRNA delivery system was named D3.

A method for combining the delivery systems prepared in Embodiment 1 and Comparative Examples 1-3 with mRNA is as follows.

Each of delivery systems prepared in Embodiment 1 and Comparative Examples 1-3 was added into an EP (Eppendorf) tube containing PBS (Phosphate Buffered Saline), and enhanced green fluorescent protein mRNA was added to an EP tube containing 100 μL of serum-free DMEM (Dulbecco's Modified Eagle Medium), followed by standing at a room temperature for 5 min. The two tubes were mixed uniformly at a mass ratio of delivery system to mRNA of 1:1, followed by standing at a room temperature for 15 minutes to obtain a desired mixture.

Samples obtained after the delivery systems prepared in Embodiment 1 and Comparative Examples 1-3 are combined with the mRNA were respectively named C1-mRNA, D1-mRNA, D2-mRNA, and D3-mRNA.

In the following experimental examples, bone marrow-derived dendritic cells (BMDCs) were obtained from the tibias and femurs of 6-10 week-old C57BL/6 mice, with culture conditions for BMDCs as follows: RPMI 1640 culture medium (10% FBS, v/v), supplemented with GM-CSF (20 ng/mL) and IL-4 (10 ng/mL), cultured in a 37° C. constant-temperature incubator (5% CO₂ and saturated humidity).

### Experimental Example 1

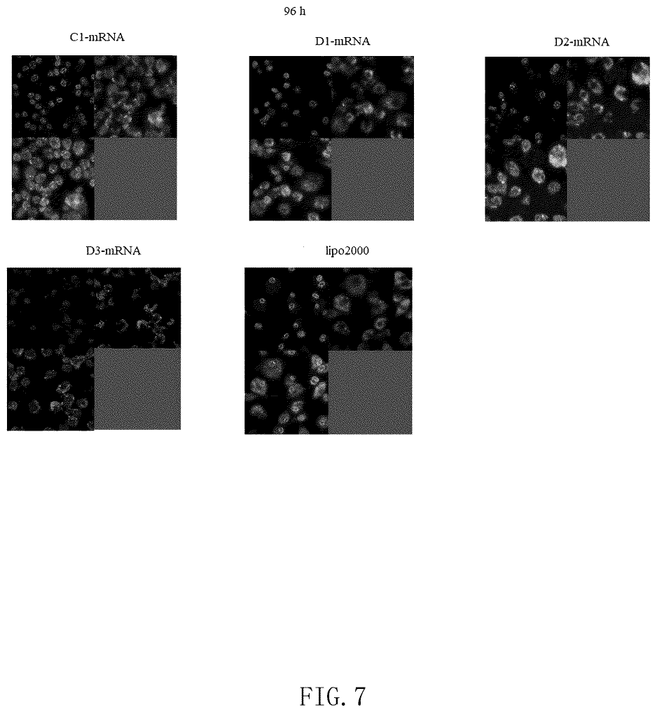

BMDC cells were seeded in a culture dish specially designed for laser confocal microscopy at a density of 3×10⁵ cells per well. 1 mL of DMEM culture medium (containing 10% FBS) was added to each well for cell culture. When the cell confluency reached 80%, C1-mRNA, D1-mRNA, D2-mRNA, and D3-mRNA were added, respectively. Multiple replicate wells were provided for each group to ensure the reliability of results. In addition, BMDC cells with Lipo 2000 served as a control group. At 96^{th} hour, the fluorescence intensity of cells in each group was observed and recorded using a laser confocal microscope. Images were captured at 400× magnification, with Master Gain of 647V for green fluorescence and 673V for blue fluorescence. The observed fluorescence changes were photographed and recorded, with results shown in FIG. 7.

As can be learned from FIG. 7 that the fluorescence intensity of the D1-mRNA and D2-mRNA groups is significantly lower than that of the Cl-mRNA group. This result shows that the delivery efficiency and targeting ability of the D1-mRNA and D2-mRNA are inferior to those of C1-mRNA, a possible reason is that in Comparative Example 1, glycocyamine is not grafted onto polyrotaxane and is just simply mixed with the polyrotaxane, and in Comparative Example 2, glycocyamine is not grafted onto the polyrotaxane. In the preparation process of the D3-mRNA group, the glycocyamine is replaced with arginine, so that the proportion of fluorescent cells in this group is significantly higher than that in the D1-mRNA and D2-mRNA groups, but still lower than that in the C1-mRNA group. The above results shows simultaneously grafting the mannose and glycocyamine onto the cyclodextrin can synergistically enhance targeting ability and delivery efficiency of the delivery system.

### Experimental Example 2

### 1. Experimental Animal

6-10 week-old female C57BL/6 mice at specific pathogen-free (SPF) grade were selected.

### 2. Grouping and Administration

The mice were randomly divided into experimental groups (Cl-mRNA group, D1-mRNA group, D2-mRNA group, D3-mRNA group), and a PBS group, with six mice in each group. The mice in the PBS group were injected with PBS solution, while the mice in the experimental groups were injected with corresponding samples, with an injection dosage of 3 mg/kg, and administration in proximity to lymph nodes.

### 3. Detection Process

Mice were sacrificed one week after injection, and peripheral blood and lymph nodes of the mice were harvested. The lymph nodes were ground into a single-cell suspension using frosted glass slide, which was then centrifuged at 350×g at 4° C. for 3 min. A supernatant was discarded, and cell pellet was collected. Hyaluronic acid (HA) was used for labeling, and Western blot (WB) was used to detect positive cells in the total protein of lymphoid tissues. The peripheral blood sample was treated with erythrocyte lysate to remove erythrocytes. After labeling with HA, the positive cells were detected by flow cytometry (such as FACSCelesta), with results shown in Table 1.

TABLE 1

Proportion of positive cells (5%)

| Group | Lymphoid tissues | Peripheral blood |
|---|---|---|
| C1-mRNA group | 80.14 | 70.94 |
| D1-mRNA group | 20.34 | 19.86 |
| D2-mRNA group | 18.62 | 13.26 |
| D3-mRNA group | 60.98 | 52.47 |
| PBS | 1.12 | 0.06 |

As can be learned from Table 1 that in the mice injected with C1-mRNA, the proportion of positive cells in the lymph nodes and peripheral blood is significantly higher than that in D1-mRNA group, D2-mRNA group and D3-mRNA group. The foregoing results show that C1 can effectively deliver mRNA to the DC cells in vivo.

### Experimental Example 3

### 1. Cell Culture

### (1) Effector Cells

Activated T cells were seeded into a 24-well plate with a density of 5×10⁵ cells/well, 1 mL of DMEM culture medium (containing 10% FBS) was added to each well to culture the cells. When the cell density reached 80%, the cells were transfected with 1 mL of Cl-mRNA (2 g/mL), 1 mL of D1-mRNA (2 g/mL), 1 mL of D2-mRNA (2 g/mL), 1 ml of D3-mRNA (2 g/mL), and 1 mL of naked mRNA (2 g/mL), respectively, the cells were collected after incubating for 48 h, centrifuging was carried out at 1500 rpm for 5 min, and a supernatant was discarded to obtain three groups of effector cells, while Cl-mRNA and naked mRNA were not added in the control group. The other methods were the same as above.

### (2) Target Cells

Bone marrow-derived dendritic cells (BMDC) were used as target cells, which were cultured in the DMEM culture medium (containing 10% FBS) to a logarithmic growth phase.

### (3) Cell Co-Culture

The concentrations of the effector cells (1×10⁵/well) and target cells (1×10⁵/well) were adjusted with the DMEM culture medium containing 10% FBS, and the effector cells and target cells were seeded into a 96-well plate with 100 L/well, which were divided into seven groups.

C1-mRNA group: T cells (transfected with C1-mRNA) were co-cultured with DC cells.

D1-mRNA group: T cells (transfected with D1-mRNA) were co-cultured with the DC cells.

D2-mRNA group: T cells (transfected with D2-mRNA) were co-cultured with the DC cells.

D3-mRNA group: T cells (transfected with D3 mRNA) were co-cultured with the DC cells.

Naked mRNA group: T cells (transfected with naked mRNA) were co-cultured with the DC cells.

Lipo 2K group: T cells (transfected with Lipo 2K) were co-cultured with the DC cells.

Control group: T cells (without being transfected) were co-cultured with the DC cells.

Cells from each group were co-cultured in triplicate at 37° C. under 5% CO₂ for 72 h. A culture supernatant was then collected, and concentrations of the cytokines IL-2 and IFN-γ were measured using an enzyme-linked immunosorbent assay (ELISA), with results shown in Table 2.

TABLE 2

| Group | IL-2 (pg/mL) | IFN-γ (pg/ml) |
|---|---|---|
| C1-mRNA group | 7500 | 4100 |
| D1-mRNA group | 3800 | 1900 |
| D2-mRNA group | 3300 | 1500 |
| D3-mRNA group | 6100 | 3100 |
| Naked mRNA group | 3100 | 1300 |
| Lipo 2000 group | 1200 | 340 |
| Control group | 110 | 190 |

As can be learned from Table 2 that the concentrations of IL-2 and IFN-r in Cl-mRNA group, D1-mRNA group, D2-mRNA group, D3-mRNA group, Lipo 2000 group and naked mRNA group are all higher than those in the control group, and the concentrations of IL-2 and IFN-γ in Cl-mRNA group are significantly higher than those in D1 to D3-mRNA groups. Therefore, a C1 carrier constructed by the present disclosure can deliver mRNA and enable high expression of mRNA in the DC cells, thereby effectively inducing T cells to produce immune response.

The foregoing are only preferred embodiments of the present disclosure, and are not limited to the above examples. For those skilled in the art, various modifications and changes can be made under the principles of the present disclosure. Any modifications, improvements and the like shall be deemed to be within the scope of protection of the present disclosure.

What is claimed is:

1. A mRNA delivery system targeting DC cells (Dendritic cells), wherein a structural formula of the delivery system is as shown in formula I: 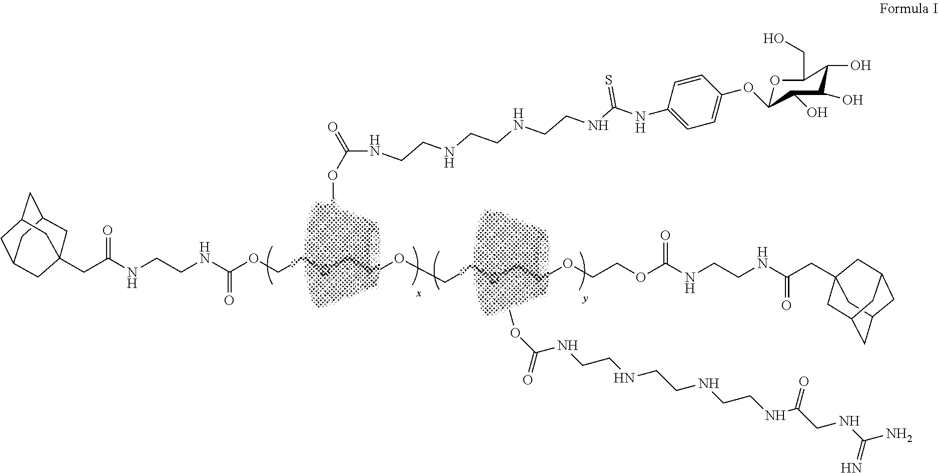
wherein x is an integer selected from 5 to 10, and y is an integer selected from 5 to 10.

2. A preparation method for the mRNA delivery system targeting DC cells according to claim 1, comprising the following steps:
(1) preparing ethylenediamine-modified polyethylene glycol: mixing polyethylene glycol with N,N′-carbonyldiimidazole, stirring at 50-55° C. for 16-20 h under nitrogen protection, and then adding ethylenediamine to react for 2-3 h, after the reaction is finished, adding ethanol to a reaction mixture, standing, collecting precipitate, washing and drying the precipitate to obtain NH₂—PEG-NH₂;
(2) preparing pseudopolyrotaxane: adding NH₂-PEG-NH₂ to a cyclodextrin aqueous solution to react, after the reaction is finished, collecting precipitate, and freeze-drying the precipitate to obtain PPR;
(3) preparing polyrotaxane: mixing PPR with a capping agent, and then adding benzotriazol-1-yloxytris(dimethylamino) phosphonium hexafluorophosphate, 1-hydroxybenzotriazole and 1,1′-[(2-hydroxyethyl)imino]dipropan-2-ol, stirring and reacting at 4-5° C. for 40-50 h under a nitrogen atmosphere, and after the reaction is finished, collecting precipitate, washing and drying the precipitate to obtain PRX;
(4) preparing urethane-modified polyrotaxane: mixing PRX with N,N′-carbonyldiimidazole, and adding triethylenetetramine for reaction, and after the reaction is finished, carrying out dialysis, and freeze-drying to obtain PRX-TETA;
(5) preparing glycocyamine-modified polyrotaxane: adding PRX-TETA to DMF (N,N-Dimethylformamide), adding Pbf-protected glycocyamine, 1-ethyl-3-(3-dimethylaminopropyl)carbodiimide hydrochloride, 2,2-bis(hydroxymethyl)propionic acid, 1-hydroxybenzotriazole and N,N-diisopropylethylamine, stirring and reacting for 12-15 h, and after the reaction is finished, centrifuging, collecting precipitate, and washing and drying the precipitate to obtain Pbf-Ga-PRX;
(6) preparing mannose-modified polyrotaxane: mixing Pbf-Ga-PRX with 4-isothiocyanatophenyl-α-D-mannoside, stirring and reacting for 24-32 h, and after the reaction is finished, carrying out dialysis, and freeze-drying to obtain Pbf-Ga-PRX-Man; and
(7) preparing a mRNA delivery system targeting DC cells: adding Pbf-Ga-PRX-Man and trifluoroacetic acid into DMF, stirring and reacting for 0.5-1 h, adding diethyl ether to a reaction system, filtering, collecting precipitate, and drying the precipitate to obtain the mRNA delivery system targeting DC cells.

3. The preparation method for the mRNA delivery system targeting DC cells according to claim 2, wherein in step (1), a mole ratio of polyethylene glycol to N,N′-carbonyldiimidazole to ethylenediamine is 1:(4.5-5):(32-33).

4. The preparation method for the mRNA delivery system targeting DC cells according to claim 2, wherein in step (2), a dosage ratio of NH₂-PEG-NH₂ to the cyclodextrin aqueous solution is 1 g:(30-40) mL; and a mass concentration of the cyclodextrin aqueous solution is 12%-13%.

5. The preparation method for the mRNA delivery system targeting DC cells according to claim 2, wherein in step (3), a mass ratio of PPR to the capping agent to the benzotriazol-1-yloxytris(dimethylamino) phosphonium hexafluorophosphate to 1-hydroxybenzotriazole is (5.5-5.6):1:(2.0-2.5):(0.7-1.0), and a dosage ratio of the capping agent to the 1,1′-[(2-hydroxyethyl)imino]dipropan-2-ol is 1 g:(0.9-1.0) mL.

6. The preparation method for the mRNA delivery system targeting DC cells according to claim 2, wherein in step (4), a mole ratio of PRX to N,N′-carbonyldiimidazole to triethylenetetramine is 1:(20-50):(50-80).

7. The preparation method for the mRNA delivery system targeting DC cells according to claim 2, wherein in step (5), a mole ratio of PRX-TETA to Pbf-protected glycocyamine to 1-ethyl-3-(3-dimethylaminopropyl)carbodiimide hydrochloride to 2,2-bis(hydroxymethyl)propionic acid to 1-hydroxybenzotriazole to N,N-diisopropylethylamine is 1:(5-10):(1-1.2):(1-1.2):(0.05-0.1):(5-5.5).

8. The preparation method for the mRNA delivery system targeting DC cells according to claim 2, wherein in step (6), a mole ratio of Pbf-Ga-PRX to 4-isothiocyanatophenyl-α-D-mannoside is 1:(5-10).

9. The preparation method for the mRNA delivery system targeting DC cells according to claim 2, wherein in step (7), a dosage ratio of Pbf-Ga-PRX-Man to trifluoroacetic acid is 1 mg:(0.9-1.5) mL.

10. The preparation method for the mRNA delivery system targeting DC cells according to claim 2, wherein the capping agent in step (3) is adamantaneacetic acid.
